(19) United States
(12) Patent Application Publication
(10) Pub. No.: US 2011/0009730 A1
(43) Pub. Date: Jan. 13, 2011

(54) MRI SYSTEM APPARATUS TO PRODUCE ELECTRIC CURRENT

(75) Inventors: Tomi Esa Kalervo Varjo, Vantaa (FI); Ilpo Asko Julius Koskela, Vantaa (FI); Mika Petri Ylihautala, Vantaa (FI)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/744,949

(22) PCT Filed: Nov. 27, 2008
(86) PCT No.: PCT/IB08/54991

§ 371 Date: Sep. 27, 2010

(30) Foreign Application Priority Data
Nov. 30, 2007 (EP) 07121979.4

(51) Int. Cl. A61B 5/055; G01R 33/36

(52) U.S. Cl. 600/410; 324/318

(57) ABSTRACT

A magnetic resonance imaging system (10) includes a main field magnet (12) which generates a B 0 magnetic field through an examination region (14). One or more electric power generators (30) are disposed in the B 0 magnetic field. Each of the electric power generators includes at least one winding or coil (34) which is rotatably mounted for movement relative to the B₀ magnetic field. A mechanical mechanism (60) such as vanes or propellers (62), a turbine (74) in conjunction with a fluid pump (72), or a motor (78) in conjunction with a drive shaft (76) and gear box (74), drive the at least one winding (34) to move in such a manner that it interacts with the B 0 magnetic field to generate an electric current.

### FIELD OF THE INVENTION

The present application relates to the magnetic resonance arts. It finds particular application in conjunction with diagnostic imaging and will be described with particular reference thereto. However, it is to be appreciated that it may also find application in conjunction with spectroscopy, radiotherapy, and the like.

### BACKGROUND OF THE INVENTION

In magnetic resonance imaging, local or surface coils are often placed directly on the patient to receive magnetic resonance signals. Electrical leads are extended from the local or surface coil to remote signal and data processing equipment. However, because these leads extend through the magnetic resonance imaging region, they are subject to RF and gradient magnetic field pulses during a magnetic resonance imaging sequence. These pulses can, in some circumstances, induce high currents or voltages on the lead lines which can be injurious to the patient.

Various proposals have been advanced to cure this problem. For example, bandpass filters have been placed along the leads. However, such filters can pass current under some circumstances.

Others have suggested using fiber optic communications. However, power still needs to be provided to the coil to operate preamplifiers, analog-to-digital converters, and other equipment for converting the analog electrical signal into an appropriate format for transmission over fiber optics.

Electrical power leads, again are susceptible to current and voltage spikes that can injure the patient or damage the electronics.

Others have proposed batteries. However, charging batteries is inconvenient and battery life can be an issue.

WO 2006/067682 proposes transferring power inductively between an RF source and the local coil.

The present application provides a new and improved method and apparatus which overcomes the above-referenced problems and others.

### SUMMARY OF THE INVENTION

In accordance with one aspect, a magnetic resonance system is provided. A magnet generates a main magnetic field, also known as B₀ magnetic field. A system induces resonance and receives induced resonance signals. An electric power generator is disposed in the B₀ field. The electric power generator includes at least one winding, a mechanism for mounting the local or surface coil for movement relative to the B₀ magnetic field, and a mechanism which drives the at least one winding to move such that it interacts with the B₀ magnetic field to generate a current.

In accordance with another aspect, an electric power generator for use in a magnetic resonance system which generates a B₀ magnetic field includes at least one winding, a mechanism for mounting the winding for movement relative to the B₀ magnetic field, and a mechanical mechanism which drives the at least one winding to move such that it interacts with the B₀ magnetic field to generate a current.

In accordance with another aspect, a magnetic resonance method is provided. A B₀ magnetic field is generated through an examination region for use in examining a subject in the examination region. At least one winding is caused to move relative to the B₀ magnetic field to generate an electric current. The generated electric current is used to power an electronic device associated with the magnetic resonance examination.

One advantage resides in an elimination of electrical connections using long conducting wires.

Another advantage resides in its wide applicability to electronics, both in and near magnetic resonance equipment.

Still further advantages and benefits will become apparent to those of ordinary skill in the art upon reading and understanding the following detailed description.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 is a diagrammatic illustration of a magnetic resonance apparatus in combination with a plurality of current generating units;

FIG. 2 is illustrative of a local receive coil mounted generator unit and associated circuitry;

FIG. 3 is illustrative of another embodiment of the electric current generator; and,

FIG. 4 is illustrative of an electric current generator which is particularly adapted for placement in a patient support or table.

### DETAILED DESCRIPTION OF EMBODIMENTS

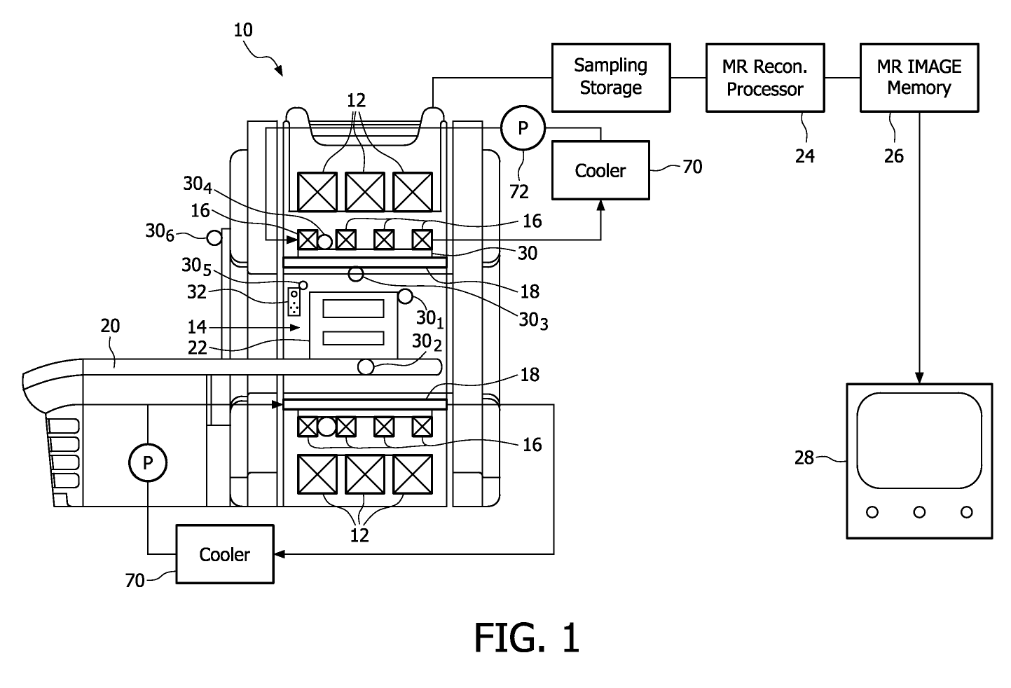

With reference to FIG. 1, a magnetic resonance apparatus 10 includes a plurality of main magnetic field coils 12, e.g., superconducting coils disposed in a dewar of a cryogenic cooling system for generating a static B₀ magnetic field through an imaging region 14. Of course, resistive magnets are also contemplated. Moreover, although a bore type scanner is illustrated, C-type scanners, open scanners, and the like can also be utilized. A gradient coil assembly 16 includes a plurality of gradient coil windings for generating gradient magnetic fields, typically in three orthogonal directions. A whole-body RF coil 18 emits radio frequency pulses for exciting and manipulating magnetic resonance in an object (a patient or other subject, for example) in the examination region 14. Optionally, the RF coil 18 can also be used as a receive coil to receive resonance signals emitted by the object following magnetic resonance excitation.

A subject or patient table or support 20 is movable longitudinally into and out of the examination region to move a region of a patient or other subject into the examination region 14. A local coil 22, such as a head coil (illustrated), knee or other joint coil, surface coil, catheter insertable coil, or the like, is moved into the examination region with the patient or subject. In some imaging sequences, resonance is excited in the examination region 14 using the whole body coil 18 and the resultant excited resonance is received by the local coil 22. The received resonance signals are reconstructed by a reconstruction processor 24 to generate one or more two- or three-dimensional magnetic resonance images for storage in an image memory 26. Human-readable images from the image representation stored in the image memory 26 are displayed on a monitor 28. Of course, other types of readout for the reconstructed image representations are also contemplated.

One or more electric current generators 30 are mounted in or near the examination region 14. In the illustrative locations of FIG. 1, an electric current generator 30₁ is mounted on the local coil 22 to supply power thereto. Another current generator 30₂ which is mounted in the patient support 20 can supply electric power, for example, to power supply jacks in the patient table into which a local coil, a patient monitor, patient entertainment device, or other power consuming device can be plugged. Short electrical leads, (e.g., less than a quarter wavelength at the resonance frequency of the nucleus being interrogated by the magnetic resonance technique), from the generator to the electronic device normally will not support an induced current or voltage pulse. Another electric current generator 30₃ is illustrated mounted in the bore and yet another generator 30₄ is illustrated mounted outside of the bore, e.g., in the inner workings of the magnetic resonance scanner, such as behind the whole body RF coil 18 adjacent the gradient coil 16. Current generators 30₃ and 30₄ may also be configured for interconnection with local coils, displays, patient entertainment, and other electronic devices. An electric current generator 30₅ is illustrated incorporated into an electronic device 32, such as a patient monitor for monitoring physiological conditions of the patient, a patient entertainment device such as an audio or video player, or the like. A current generator 30₆ is illustrated mounted outside of the bore but still within the main magnetic field. The electric current generators 30 can also be used to provide power for monitoring patient table position, for fMRI probes, and the like.

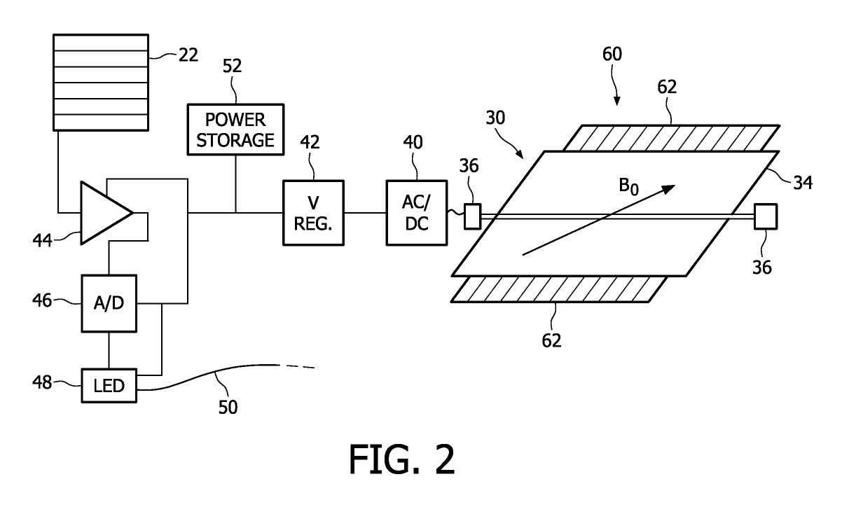

With reference to FIG. 2, the current generators 30 each include a coil winding 34 which is mounted on bearings 36 for rotation relative to the B₀ field. As the windings cut the B₀ field, currents are generated in the windings. Various winding patterns such as loops, figure-of-8s, toroids, and the like as are known in the art for generating electrical current are contemplated. If the required current is DC, the windings are connected with an AC to DC converter 40 and a voltage regulator 42. The voltage regulator 42 provides a voltage of the appropriate magnitude for electronic components which it is to power. In the example of FIG. 2, windings of the local coil 22 are connected with an amplifier 44 which is powered by voltage from the voltage regulator 42. The amplified signal received by the local coil is converted to a digital signal by an analog-to-digital converter 46 and to an optical signal by an electro/optical converter 48. The resultant digital optical signal is conveyed by an optic fiber 50 to the magnetic resonance reconstruction processor 24 (shown in FIG. 1) or other remote electronics. Of course, the exact nature of the electronics powered by the generated electric current or voltage will vary with the application. Optionally, an electric power storage device 52, such as a capacitor or rechargeable battery is connected with the voltage regulator. The electric power storage device can be charged when power is not used or fully consumed by the electronic components and discharged to supply power when needed. In this manner, a smaller electric current generator can be utilized for equipment that uses power in spurts.

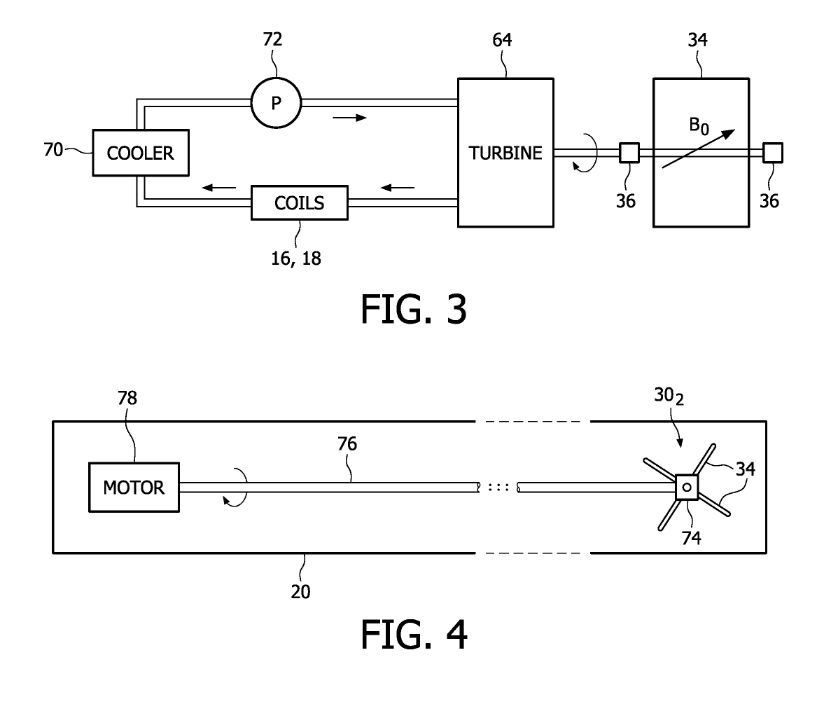

Various mechanisms 60 are contemplated for driving the winding 34 to rotate relative to the B₀ field. In the embodiment of FIG. 2, the mechanism includes blades or propellers 62 which are propelled by passing air to rotate the windings 34. The moving air can be supplied by the scanner's ventilation system or a supplemental fan. In the embodiment of FIG. 3, the mechanism 60 for rotating the windings 34 includes a fluid turbine 64.

As illustrated in FIG. 1, a fluid cooler 70 is connected to a pump 72 which pumps a cooling fluid through the gradient coil 16, the RF coils 18, or other components of the magnetic resonance system 10. The cooling fluid is pumped not only through the components of the scanner 10, but also through the turbine 64 to be converted into rotational force to rotate the windings 34. Cooling fluids may include liquids, gases, or the like. If the cooling fluid is air, then the air need not be recirculated through a cooler. Rather, the air can be pumped from the ambient atmosphere and discharged to the ambient atmosphere. The cooling fluid may also be used to cool other components, such as a PET detector in a combined PET/MR imaging system. Although FIGS. 1 and 3 illustrate an embodiment in which the fluid which drives the turbines 64 performs the dual functions of driving the turbine and providing cooling, it is contemplated that the fluid may be provided for the sole purpose of driving the turbine 64 of one or more current generators 30.

In another embodiment, the current generator 30, such as the current generator 30₂ disposed in the patient bed 20 is driven by a mechanical linkage. The rotatable windings 34 are interconnected by a gear box 74 to a rotating drive shaft 76. The drive shaft extends through the patient support or other structure of the MR magnetic resonance scanner 10 to a drive motor 78. The drive motor 78, such as an electric motor, pneumatic motor, hydraulic motor, or the like, can be disposed outside of the examination region 14 for convenience of access. Rather than a drive shaft, a chain drive, a belt drive or other mechanical linkage can also be utilized.

Numerous other mechanisms which provide the motive power to rotate the windings through the B₀ field to generate electric power as are known to those of ordinary skill in the art are also contemplated. Moving the windings in other than a circular rotation pattern is also contemplated.

Because the current generators 30 can be disposed in or near the imaging region 14, they are preferably constructed of materials which are not imaged by the magnetic resonance imaging system or which do not interfere with the B₀ magnetic fields, the gradient magnetic fields, or the RF fields sufficiently to cause image distortion or degradation. Various magnetic resonance inert materials such as aluminum, stainless steel, various plastics which do not resonate near the resonance frequency of the magnetic resonance scanner 10, dielectric oils, and the like are contemplated. To avoid interference with RF signals in the magnetic resonance imaging sequence, the current generators 30 advantageously are shielded with a Faraday or RF shield.

The invention has been described with reference to the preferred embodiments. Modifications and alterations may occur to others upon reading and understanding the preceding detailed description. It is intended that the invention be construed as including all such modifications and alterations insofar as they come within the scope of the appended claims or the equivalents thereof. In the claims, any reference signs placed between parentheses shall not be construed as limiting the claim. The word “comprising” does not exclude the presence of elements or steps other than those listed in a claim. The word “a” or “an” preceding an element does not exclude the presence of a plurality of such elements. The disclosed method can be implemented by means of hardware comprising several distinct elements, and by means of a suitably programmed computer. In the system claims enumerating several means, several of these means can be embodied by one and the same item of computer readable software or hardware. The mere fact that certain measures are recited in mutually different dependent claims does not indicate that a combination of these measures cannot be used to advantage.

1. A magnetic resonance system comprising:
a magnet which generates a B₀ magnetic field;
a system for inducing resonance and receiving induced resonance signals;
an electric power generator disposed in the B₀ magnetic field, the electric power generator including:
at least one winding,
a mechanism for mounting the winding for movement relative to the B₀ magnetic field,
a mechanical mechanism which drives the at least one winding to move such that it interacts with the B₀ magnetic field to generate a current.

2. The system according to claim 1, wherein the mechanical mechanism includes fan or impeller blades.

3. The system according to claim 1, wherein the mechanical mechanism includes a fluid-driven turbine.

4. The system according to claim 1, wherein the drive mechanism includes a mechanical drive.

5. The system according to claim 1, wherein the system for inducing resonance and receiving induced resonance signals includes a local receive coil, the electric power generator being mounted to at least one of the local receive coil or a patient support adjacent to the local receive coil.

6. The system according to claim 5, wherein the local receive coil includes at least one of:
an amplifier connected with the electric current generator to receive operating power therefrom; and/or
an analog-to-digital converter connected with the electric current generator to receive operating power therefrom.

7. The system according to claim 1, further including an electronic device configured for selective deployment into the B₀ field, the current generator being coupled with the electronic device to provide operating power thereto.

8. The system according to claim 7, wherein the electronic device includes at least one of a physiological monitor, a patient entertainment device, a music player, a video player, an fMRI probe, or a patient table position monitor.

9. The system according to claim 1, further including:
a subject support for moving a subject into and out of an examination region, the electric current generator being mounted in the subject support.

10. The system according to claim 1, further including:
a fluid cooling system which circulates cooling fluid to heat-generating components and wherein the current generator drive mechanism includes a fluid-driven turbine connected with the fluid cooling system.

11. The system according to claim 1, wherein the magnetic resonance system is a magnetic resonance imaging system which generates magnetic resonance images of a portion of a subject in an examination region and wherein the current generator is disposed outside of the examination region and in the B₀ magnetic field.

12. An electric generator for use in a magnetic resonance system which generates a B₀ magnetic field, the electric generator comprising:
at least one winding;
a mechanism for mounting the winding for movement relative to the B₀ magnetic field; and
a mechanical mechanism which drives the at least one winding to move such that it interacts with the B₀ magnetic field to generate a current.

13. A magnetic resonance method comprising:
generating a B₀ magnetic field through an examination region for use in a magnetic resonance examination of a subject in the examination region; and
causing at least one winding in the B₀ magnetic field to move relative to the B₀ magnetic field to generate an electric current.

14. The method according to claim 13, including using the generated electric current to power an electronic device associated with the magnetic resonance examination.

15. The method according to claim 13, wherein causing the winding to move includes driving the winding to rotate with at least one of flowing fluid or a mechanical mechanism.
